(19) United States
(12) Patent Application Publication
(10) Pub. No.: US 2002/0120956 A1
(43) Pub. Date: Aug. 29, 2002

(54) Resistance to gummy stem blight in melon

(76) Inventor: Margaret M. Jahn, Lansing, NY (US)

Correspondence Address: Michael L. Goldman, Esq., NIXON PEABODY LLP, Clinton Square, P.O. Box 31051, Rochester, NY, 14603-1051, US

(21) Appl. No.: 09/993,856

(22) Filed: Nov. 14, 2001

Related U.S. Application Data
(60) Provisional application No. 60/248,481, filed on Nov. 14, 2000.

(51) Int. Cl. A01H 5/00

(52) U.S. Cl. 800/279; 800/309

(57) ABSTRACT

The present invention is directed to a method for producing gummy stem blight resistant Cucumis melo hybrid seeds, plants, breeding lines, pollen, ovules, and tissue culture. The method involves using resistant C. melo plants as sources of gummy stem blight resistance in plant breeding programs. Also disclosed are the gummy stem blight resistant C. melo seeds, plants, breeding lines, pollen, tissue culture, and ovules produced using the methods of the present invention.

This application claims the benefit of U.S. Provisional Patent Application Serial No. 60/248,481, filed Nov. 14, 2000.

The subject matter of this application was made with support from the United States Government under United States Agency for International Development (USAID) Contract No. DAN-A-00-91-0000126-00. The U.S. Government may have certain rights.

### FIELD OF THE INVENTION

The present invention relates to hybrid Cucumis melo plants having resistance to gummy stem blight as well as the seeds, ovules, pollen, tissue culture, and other parts of these hybrid plants, and the methods for producing these plants.

### BACKGROUND OF THE INVENTION

Melons are members of the Cucurbitaceae family and belong to the Cucumis melo L. species (Munger, “Breeding for Viral Disease Resistance in Cucurbits,” in Kyle, ed., Resistance to Viral Diseases in Vegetables: Genetics and Breeding Portland, Oreg.: Timber Press, pp. 44-60, at pp. 53-54 (1993)). Melons have been divided into a number of groups, based on the phenotypic and morphologic differences within the C. melo species. One commonly recognized grouping of melons includes the following groups: (1) C. melo agrestis Naud. (e.g., wild melons with inedible fruits); (2) C. melo cantalupensis Naud. (e.g., cantaloupe, muskmelon); (3) C. melo indorus Naud. (e.g., winter melon); (4) C. meloflexuousus (e.g., snake melon); (5) C. melo conomon (e.g., pickling melon, sweet melon); (6) C. melo chito/C. melo dudaim Naud. (e.g., mango melon); and (7) C. melo momordica (e.g., snap melon) (Munger et al., “Nomenclature of Cucumis melo L.,” Cucurbit Genetics Cooperative Report 14:43-44 (1991)). In the present application, the term “melon” is used interchangeably with C. melo.

In terms of commercial importance, the three most important melon groups are C. melo cantalupensis, C. melo indorus, and C. meloflexuousus. These three groups may be crossed with one another, themselves, or other melon groups to produce seeds that develop into melon plants with characteristics from more than of one of the melon groups.

The C. melo cantalupensis group is not grown commercially in the United States to any great extent. This group is referred to as the “true cantaloupe.” This group includes the melons commonly referred to as cantaloupe and muskmelon. These melons have medium size fruits with netted, warty, or scaly surfaces. Their flesh are usually orange, but sometimes green, and their flavor is aromatic or musky. Their fruit are dehiscent at maturity, and they are usually andromonoecious. This group includes melons that have been grouped into the C. melo reticulatus group (Munger et al., “Nomenclature of Cucumis melo L.,” Cucurbit Genetics Cooperative Report 14:43-44 (1991)). The C. melo reticulatus group has been described to include the melons having a netted surface.

The C. melo inodorus group is referred to as winter melon and includes such melons as honeydew, casaba, Crenshaw, Santa Claus melon, Persian melon, and Juan Canary. The honeydew melon is the most important melon in commercial terms. These melons appear smooth or wrinkled on their surface and have white or green flesh. They lack any musky odor. As compared to melons of the C. melo cantalupensis group, melons of the C. melo indorus group are usually larger, later to mature, and longer-keeping. They are not dehiscent at maturity. They are usually andromonoecious (Munger et al., “Nomenclature of Cucumis melo L.,” Cucurbit Genetics Cooperative Report 14:43-44 (1991)).

Melons in the C. meloflexuousus group are commonly referred to as snake melons. Their fruit are long and slender and are used at their immature stage as an alternative to cucumber. These melons are monoecious.

Gummy stem blight (“GSB”) is one of the most serious diseases of melon in the United States and throughout the world. The disease is caused by the fungus Didymella bryoniae (Auersw.) Rehm (synonyms: Mycosphaerella citrullina (C. O. Sm.) Gross. and M. melonis (Pass.) Chiu & J. C. Walker) and its anamorph Phoma cucurbitacearum (Fr:Fr.) Sacc. (synonyms: Asochyta cucumis Fautrey & Roum. and A. citrullina (F. Chester) C. O. Sm.) (Sherf et al., Vegetable Diseases and Their Control, New York: Wiley-Interscience, pp. 340-346 (1986)). The disease is most common in tropical and sub-tropical areas of the world, but it is also a serious disease of cucurbits in the United States (Sitterly et al., “Gummy Stem Blight,” in Zitter et al., eds., Compendium of Cucurbit Diseases, St. Paul, Minn..: APS Press, pp. 27-28 (1996)). In the southeastern states, GSB is the most destructive disease of watermelon (Citrullus lanatus) and cucumber (Cucumis sativus) (Keinath, A. P., “Fungicide Timing for Optimum Management of Gummy Stem Blight Epidemics on Watermelon,” Plant Dis. 79:354-358 (1995); and St. Amand et al., “Crop Loss to 14 Diseases of Cucumber in North Carolina from 1983 to 1988,” Cucurbit Genet. Coop. Rep. 14:15-17 (1991)), and also causes serious losses in melon. The disease has increased in importance in recent years as effective chemical and genetic control of other cucurbit diseases has become available (Sitterly, W. R., “Breeding for Disease Resistance in Cucurbits,” Annu. Rev. Phytopathol. 10:471-490 (1972)).

Symptoms of GSB include circular, tan to dark-brown spots on leaves that may enlarge under favorable conditions to cover the leaf. On cotyledons and stems of young plants, circular black or tan lesions may be evident. Water-soaked areas may develop on hypocotyls and leaves. Cankers may appear in stem cortical tissue that produce a typical brown, gummy exudate and may girdle the stem, resulting in plant death (Sitterly, W. R., “Breeding for Disease Resistance in Cucurbits,” Annu. Rev. Phytopathol. 10:471-490 (1972)).

Control practices include the use of fungicide-treated seed and a minimum two-year crop rotation (Sitterly et al., “Gummy Stem Blight,” in Zitter et al., eds., Compendium of Cucurbit Diseases, St. Paul, Minn.: APS Press, pp. 27-28 (1996)). Although satisfactory chemical control can be achieved with fungicides (Keinath, A. P., “Fungicide Timing for Optimum Management of Gummy Stem Blight Epidemics on Watermelon,” Plant Dis. 79:354-358 (1995)), resistance to benzimidazole products has been reported (Keinath et al., “Control of Gummy Stem Blight in Watermelon with Fungicides, 1991,” Fungic. Nematic. Tests 47:156 (1992); Keinath et al., “First Report on Benomyl-Insensitive Didymella bryoniae in the United States.,” (Abstr.) Phytopathology 85:1126 (1995); Keinath et al., “Resistance to Benomyl and Thiophanate-Methyl in Didymella bryoniae from South Carolina and New York,” Plant Dis. 82:479-484 (1998); and Sitterly et al., “Gummy Stem Blight,” in Zitter et al., eds., Compendium of Cucurbit Diseases, St. Paul, Minn.: APS Press, pp. 27-28 (1996)).

Several sources of GSB resistance in wild C. melo accessions from the United States Department of Agriculture (U.S.D.A.) National Plant Germplasm System (N.P.G.S.) have been previously reported (McGrath et al., “Resistance to Gummy Stem Blight in Muskmelon,” HortScience 28:930-931 (1993); Sowell, G., Jr., “Additional Sources of Resistance to Gummy Stem Blight of Muskmelon,” Plant Dis. 65:253-254 (1981); Sowell et al., “Resistance of Cucumis melo Introductions to Mycosphaerella citrullina,” Plant Dis. Rep. 50:661-663 (1996); and Zhang et al., “Screening Melon (Cucumis melo) for Resistance to Gummy Stem Blight in the Greenhouse and Field,” HortScience 32:117-121 (1997)).

A recent study that employed both greenhouse and field evaluations yielded several new sources of resistance to GSB among the 800 accessions examined. Levels of resistance identified in this study were equal to or greater than any previously reported (Zhang et al., “Screening Melon (Cucumis melo) for Resistance to Gummy Stem Blight in the Greenhouse and Field,” HortScience 32:117-121 (1997)). Although genetic resistance was identified in C. melo in the 1960s and efforts to incorporate resistance into cultivated melon have been ongoing (Norton, J. D., “Gulfcoast—A Sweet Cantaloupe for the Produce Chain Store Market,” Ala. Agric. Exp. Stn. Leafl. 82 (1971); Norton, J. D., “Chilton, a High Quality Fruit for the Commercial Market,” Ala. Agric. Exp. Stn. Leafl. 84 (1972); Norton et al., “Ac-70-154 a Gummy Stem Blight-Resistant Muskmelon Breeding Line,” HortScience 24:709-711 (1989); Norton et al., “Aurora—A High Quality Disease Resistant Cantaloupe,” Ala. Agric. Exp. Stn. Circ. 278 (1985)), no commercial varieties with adequate levels of resistance are currently available (Sitterly et al., “Gummy Stem Blight,” in Zitter et al., eds., Compendium of Cucurbit Diseases, St. Paul, Minn.: APS Press, pp. 27-28 (1996)). Thus, there is still a need to incorporate higher levels of genetic resistance in melon varieties.

The present invention is directed to overcoming these deficiencies in the prior art.

### SUMMARY OF THE INVENTION

The present invention relates to a method for producing a gummy stem blight resistant Cucumis melo hybrid seed. This method involves crossing a first C. melo plant with a second C. melo plant to yield a first generation hybrid seed. The first C. melo plant is either resistant or not resistant to gummy stem blight, while the second C. melo plant is resistant to gummy stem blight.

The present invention also relates to a gummy stem blight resistant C. melo hybrid seed, prepared by crossing a first C. melo plant with a second C. melo plant, to yield a first generation hybrid seed. The first C. melo plant is either resistant or not resistant to gummy stem blight, while the second C. melo plant is resistant to gummy stem blight.

The present invention also relates to a gummy stem blight resistant C. melo hybrid plant, prepared by crossing a first C. melo plant with a second C. melo plant to yield a first generation hybrid seed, and then growing the first generation hybrid seed to yield a first generation resistant C. melo hybrid plant. The first C. melo plant is either resistant or not resistant to gummy stem blight, while the second C. melo plant is resistant to gummy stem blight.

The present invention also relates to seed of a gummy stem blight resistant C. melo breeding line designated NY 01-190-3R, -7L, -9L (composite), a sample of the seed having been deposited under ATCC accession number ______.

The present invention also relates to pollen, ovules, seeds, and tissue culture derived from the resistant C. melo seeds and plants produced according to the methods of the present invention.

The present invention is useful in developing commercially appealing C. melo hybrid plants and plant parts that are resistant to gummy stem blight.

The present invention uses traditional plant breeding techniques to develop GSB resistant melon plants lines that are commercially appealing. One goal of plant breeding is to combine in a single variety or hybrid various desirable traits. These traits may include resistance to diseases and insects, resistance to herbicides, resistance to pesticides, higher seed yield, better quality fruit, better stems and roots, improved compositional traits, tolerance to heat and drought, reduced maturity time, greater yield, and better agronomic quality.

Generally, development of a GSB resistant melon variety or hybrid line would involve (1) identifying melon accessions suitable for use as GSB resistance source plants and (2) using the identified GSB resistance source plants in breeding programs to yield GSB resistant progeny that are commercially appealing.

Popular selection methods commonly used in such breeding programs include, for example, pedigree selection, modified pedigree selection, mass selection, recurrent selection, and backcrossing. The complexity of inheritance influences choice of the breeding method. Generally, backcross breeding is used to transfer one or a few favorable genes for a highly heritable trait into a desirable cultivar. This approach has been used extensively for breeding disease-resistant cultivars (Nickell et al., “Registration of L84-5873 and L84-5932 Soybean Germplasm Lines Resistant to Brown Stem Rot,” Crop Sci. 32:835 (1992), which is hereby incorporated by reference in its entirety). Various recurrent selection techniques are used to improve quantitatively inherited traits controlled by numerous genes.

The present invention also uses tissue culture techniques in breeding programs to develop GSB resistant melon plant lines that are commercially appealing.

### DETAILED DESCRIPTION OF THE INVENTION

The present invention relates to a method for producing a gummy stem blight (“GSB”) resistant Cucumis melo hybrid seed. This method involves crossing a first C. melo plant with a second C. melo plant to yield a first generation hybrid seed. The first C. melo plant is either resistant or not resistant to gummy stem blight, while the second C. melo plant is resistant to gummy stem blight.

The present invention also relates to a gummy stem blight resistant C. melo hybrid seed, prepared by crossing a first C. melo plant with a second C. melo plant, to yield a first generation hybrid seed. The first C. melo plant is either resistant or not resistant to gummy stem blight, while the second C. melo plant is resistant to gummy stem blight.

The present invention also relates to a gummy stem blight resistant C. melo hybrid plant, prepared by crossing a first C. melo plant with a second C. melo plant to yield a first generation hybrid seed, and then growing the first generation hybrid seed to yield a first generation resistant C. melo hybrid plant. The first C. melo plant is either resistant or not resistant to gummy stem blight, while the second C. melo plant is resistant to gummy stem blight.

The present invention also relates to seed of a gummy stem blight resistant C. melo breeding line designated NY 01-190-3R, -7L, -9L (composite), a sample of the seed having been deposited under ATCC accession number ______.

The present invention also relates to pollen, ovules, seeds, and tissue culture derived from the resistant C. melo seeds and plants produced according to the methods of the present invention.

The present invention is useful in developing commercially appealing C. melo hybrid plants and plant parts that are resistant to gummy stem blight.

The term “allele” refers to any of one or more alternative forms of a gene locus, all of which alleles relate to one trait or characteristic. In a diploid cell or organism, the two alleles of a given gene occupy corresponding loci on a pair of homologous chromosomes.

The term “andromonoecious” refers to plant species that bear both male and bisexual (hermaphrodite) flowers on the same plant. (Rudin, Dictionary of Modern Biology Hauppauge, N.Y.: Barron's p. 16 (1997), which is hereby incorporated by reference in its entirety.)

The term “backcrossing” refers to a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “crossing” refers to the mating of two parent plants.

The term “dioecious” refers to plants in which male and female flowers are borne on separate individuals. (Rudin, Dictionary of Modern Biology Hauppauge, N.Y.: Barron's p. 111 (1997), which is hereby incorporated by reference in its entirety.)

The term “diploid” refers to a cell or organism having two sets of chromosomes.

The term “F1 hybrid” refers to the first generation progeny of the cross of two nonisogenic plants.

The term “genotype” refers to the genetic constitution of a cell or organism.

The term “monoecious” refers to plants in which male and female reproductive organs are borne on separate flowers but on the same individual. (Rudin, Dictionary of Modern Biology Hauppauge, N.Y.: Barron's p. 236 (1997), which is hereby incorporated by reference in its entirety.)

The term “phenotype” refers to the detectable characteristics of a cell or organism, which characteristics are the manifestation of gene expression.

The term “plant” refers to whole plants, plant cells, plant protoplasts, plant cells of a tissue culture from which melon plants can be regenerated, plant calli, plant clumps, and plant cells that are intact in plants or parts of plants, such as pollen, flowers, seeds, fruit, leaves, stems, and the like.

The term “self-pollination” refers to the transfer of pollen from the anther to the stigma of the same plant.

The term “single locus converted (conversion) plant” refers to plants which are developed by a plant breeding technique called backcrossing, wherein essentially all of the desired morphological and physiological characteristics of a melon cultivar are recovered in addition to the characteristics of the single locus transferred into the cultivar via the backcrossing technique.

The term “tissue culture” refers to a composition comprising isolated cells of the same or a different type or a collection of such cells organized into parts of a plant.

The terms “resistance” and “resistant,” as applied to the melon plants, or their parts, of the present invention, generally describe a phenotypic trait characterized by reduction in size and extent of spread of foliar and stem lesions, delay of vine death, and reduction of yield loss due to the gummy stem blight pathogen.

The present invention also relates to GSB-resistant C. melo hybrid plants, seeds, and plant parts, where the non-resistant C. melo cultivars used to produce the resistant hybrid plants, seeds, or plant parts (e.g., pollen, ovules, tissue culture) are from the following groups: C. melo cantalupensis Naud. (e.g., cantaloupe, muskmelon); C. melo indorus Naud. (e.g., winter melon); and C. melo flexuousus (e.g., snake melon) (Munger et al., “Nomenclature of Cucumis melo L.,” Cucurbit Genetics Cooperative Report 14:43-44 (1991), which is hereby incorporated by reference in its entirety). Other groups of melon that may be used in the present invention as non-resistant cultivars include the following: C. melo agrestis Naud. (e.g., wild melons with inedible fruits); C. melo conomon (e.g., pickling melon, sweet melon); C. melo chito/C. melo dudaim Naud. (e.g., mango melon); and C. melo momordica (e.g., snap melon) (Munger et al., “Nomenclature of Cucumis melo L.,” Cucurbit Genetics Cooperative Report 14:43-44 (1991), which is hereby incorporated by reference in its entirety).

The present invention also relates to methods and compositions relating to plants, seeds, and other plant parts (e.g., pollen, ovules, tissue culture), where the resistant C. melo cultivars used to produce the resistant hybrid plants, seeds, or plant parts are from the following GSB resistant U.S.D.A. Plant Introduction (“PI”) melon accessions: PI 157082 (isolated from China); PI 511890 (isolated from Mexico); PI 482399 (isolated from Zimbabwe); PI 482398 (isolated from Zimbabwe); and PI 140471 (isolated from Texas), collectively referred to herein as the “GSB Resistant Melons.”

The present invention also involves GSB-resistant C. melo hybrid plants, seeds, and plant parts, where the non-resistant C. melo cultivars used to produce the resistant hybrid plants, seeds, or plant parts (e.g., pollen, ovules, tissue culture) are from, without limitation, the following non-resistant cultivars: Cornell ZPPM 339, TAM Uvalde, UC Topmark, Galia type, Ananas type, and Oro Rico.

The present invention also relates to at least one monogenic, dominant GSB resistance gene as a source of GSB resistance in the plant breeding programs of the present invention. One embodiment involves, without limitation, the following GSB dominant resistance gene loci: Gsb1 (also referred to in the art as the MC gene locus), Gsb2, Gsb4, and Gsb5.

The present invention also relates to at least one monogenic, recessive GSB resistance gene as a source of GSB resistance in the plant breeding program of the present invention. One embodiment involves, without limitation, the gsb3 resistance gene.

The present invention can involve combining at least two, distinct GSB resistance gene loci into a single melon hybrid cultivar or variety in order to achieve enhanced GSB resistance in the hybrid. This can involve the combination of at least one monogenic dominant GSB resistance gene with at least one monogenic recessive GSB resistance gene in a single hybrid melon cultivar. Alternatively, at least one monogenic dominant GSB resistance gene is combined with at least one other distinct monogenic dominant GSB resistance gene in a single hybrid melon cultivar. In yet another embodiment, at least one monogenic recessive GSB resistance gene is combined with at least one other distinct recessive GSB resistance gene in a single hybrid melon cultivar. The monogenic dominant GSB resistance genes used for this aspect of the present invention may include, without limitation, the Gsb1 (=MC), Gsb2, Gsb4, and Gsb5 genes. The monogenic recessive GSB resistance genes used for this aspect of the present invention may include, without limitation, the gsb1 gene. These five monogenic GSB resistance genes are separate and distinct from one another, and are from the following PI accessions: Gsb1, from PI 140471; Gsb2, from PI 157082; gsb3, from PI 482399; Gsb4, from PI 511890; and Gsb5, from PI 482398.

The combination of distinct resistance genes for the same pathogen, but on different gene loci, into a single hybrid cultivar is commonly referred to in the art as “pyramiding.”

GSB resistance derived from the breeding programs of the present invention utilize breeding techniques that are well known in the art, including, without limitation, selfing, recurrent selection, backcrossing, pedigree breeding, restriction length polymorphism enhanced selection, genetic marker enhanced selection, and transformation.

Single locus conversions of GSB melon hybrids, including those hybrids deriving GSB resistance from the GSB Resistant Melons, can also be used. The term single locus converted plant as used here refers to those melon plants which are developed by a plant breeding technique called backcrossing, where essentially all of the desired morphological and physiological characteristics of a commercially desirable melon cultivar are recovered in addition to the single locus (e.g., a GSB resistance gene) transferred into the cultivar via the backcrossing technique. Backcrossing methods can be used with the present invention to improve or introduce a characteristic into the present cultivar. The term backcrossing as used here refers to the repeated crossing of a hybrid progeny back to one of the parental melon plants for that hybrid. The parental melon plant which contributes the locus for the desired characteristic is termed the nonrecurrent or donor parent. This terminology refers to the fact that the nonrecurrent parent is used one time in the backcross protocol and, therefore, does not recur. The parental melon plant to which the locus or loci from the nonrecurrent parent are transferred is known as the recurrent parent as it is used for several rounds in the backcrossing protocol (Poehlman, J. M. & D. A. Sleper, Breeding Field Crops, 4th Ed., Ames, Iowa: Iowa State University Press. Ames, Iowa (1995); Fehr, W. R., ed., Principles of Cultivar Development, Vol. 1: Theory and Technique, New York, N.Y.: Macmillan Publishing Company (1987); and Fehr, W. R., ed., Principles of Cultivar Development, Vol. 2: Crop Species, New York, N.Y.: Macmillan Publishing Company (1987), which are hereby incorporated by reference in their entirety).

In a typical backcross protocol, the phenotypically and/or commercially appealing cultivar or accession (recurrent parent) is crossed with a second cultivar (nonrecurrent parent) that carries the single locus of interest (e.g., the GSB resistance gene locus) to be transferred. The resulting progeny from this cross are then crossed again to the recurrent parent and the process is repeated until a melon plant is obtained where essentially all of the desired morphological and physiological characteristics of the recurrent parent are recovered in the converted plant, in addition to the single transferred locus from the nonrecurrent parent.

The selection of a suitable recurrent parent is an important step for a successful backcrossing procedure. The goal of a backcross protocol is to alter or substitute a single trait or characteristic in the original cultivar or accession. To accomplish this, a single locus of the recurrent cultivar is modified or substituted with the desired locus from the nonrecurrent parent, while retaining essentially all of the rest of the desired genetic, and therefore the desired physiological and morphological constitution of the original cultivar. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Melon cultivars can also be developed from more than two parents (Fehr, W. R., ed., Principles of Cultivar Development, Vol. 1: Theory and Technique, New York, N.Y.: Macmillan Publishing Company (1987), which is hereby incorporated by reference in its entirety). The technique, known as modified backcrossing, uses different recurrent parents during the backcrossing. Modified backcrossing may be used to replace the original recurrent parent with a cultivar having certain more desirable characteristics or multiple parents may be used to obtain different desirable characteristics from each.

Many single locus traits have been identified that are not regularly selected for in the development of a new inbred but that can be improved by backcrossing techniques. Single locus traits may or may not be transgenic; examples of these traits include, but are not limited to, male sterility, herbicide resistance, disease resistance, insect resistance, enhanced nutritional quality, yield stability, and yield enhancement. These comprise genes that are generally inherited through the nucleus.

Direct selection may be applied where the single locus acts as a dominant trait. In the present invention, PI 157082, PI 511890, PI 482398, and PI 140471 were used as sources of single, dominant GSB resistance genes. For this selection process, the progeny of the initial cross are exposed to the GSB pathogen, using methods well known in the art, prior to the backcrossing. This step allows one to identify and eliminate any plants which do not have the desired GSB resistance characteristic, and, thus, only those plants which have the GSB resistance gene are used in the subsequent backcross. This process is then repeated for all additional backcross generations.

In another embodiment of the present invention, the development of GSB resistant melon hybrids in a plant breeding program involves the development of homozygous inbred lines of GSB resistant melons, the crossing of these lines, and the evaluation of the crosses. Pedigree breeding and recurrent selection breeding methods are used to develop inbred lines from breeding populations. This type of plant breeding program combines the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Examples of potentially desired characteristics in melon include, without limitation, enhanced seed yield, fruit size, fruit quality, fruit shelf life, seedling vigor, disease and insect tolerance, and maturity rate.

A GSB resistant melon can be crossed with itself or a second plant and the seeds and plants and germplasm produced by such methods. These methods can be used for propagation of the GSB resistant melon cultivars of the present invention, or can be used to produce hybrid melon seeds and the plants grown therefrom. A hybrid melon plant can also be used as a recurrent parent at any given stage in a backcrossing protocol during the production of a single locus conversion of the melon cultivars of interest.

Any time a GSB resistant melon cultivar, including without limitation, the GSB Resistant Melon accessions of the present invention, are crossed with a different melon cultivar, first generation (F1) melon progeny are produced. The hybrid progeny are produced regardless of characteristics of the two cultivars produced. As such, an F1 hybrid melon plant may be produced by crossing a GSB resistant melon cultivar with any second melon plant. The second melon plant may be genetically homogeneous (e.g., inbred) or may itself be a hybrid. Therefore, any F1 hybrid melon plant produced by crossing a GSB resistant melon cultivar with a second melon plant is a part of the present invention.

Recurrent selection breeding, backcrossing for example, can be used in conjunction with the present invention to improve an inbred line and a hybrid which is made using those inbreds. Backcrossing can be used to transfer a specific desirable trait from one inbred or source to an inbred that lacks that trait. For example, this can be accomplished by first crossing a superior inbred (recurrent parent) to a donor inbred (non-recurrent parent), that carries the appropriate gene(s) for the trait in question. The recurrent parent typically includes many commercially important traits, while the non-recurrent parent includes a resistance trait, as is the case for the gummy stem blight resistant melons. The progeny of this cross are then mated back to the superior recurrent parent followed by selection in the resultant progeny for the desired trait to be transferred from the non-recurrent parent. After five or more backcross generations with selection for the desired trait, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

GSB resistant melons, including without limitation, the GSB Resistant Melons, can be used in a pedigree breeding program. Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to homogeneous lines as a result of self-pollination and selection. Typically, five or more generations of selfing and selection is practiced: F1→F2; F2→F3; F3→F4; F4→F5, etc. Variations of this generalized pedigree method are used, but all these variations produce a segregating generation which contains a range of variation for the traits of interest.

Thus, in general terms, pedigree breeding involves crossing two inbred lines to produce the non-segregating F1 generation, and self pollination of the F1 generation to produce the F2 generation that segregates for all factors for which the inbred parents differ. A hypothetical example of this process is set forth below.

Hypothetical Example of Pedigree Breeding Program:

Cross between two inbred lines that differ for alleles at six loci. The parental genotypes are:

Parent 1 AbCdeF/AbCdeF

Parent 2 aBcDEf/aBcDEf,

and the F1 from a cross between these two parents is:

F1 AbCdeF/aBcDEf

Selfing F1 will produce an F2 generation including the following genotypes:

F2 ABcDEf/abCdeF

F2 ABcDef/abCdEF

F2 ABcDef/abCdeF.

Each inbred parent which is used in breeding crosses represents a unique combination of genes, and the combined effects of the genes define the performance of the inbred and its performance in hybrid combination. There is published evidence (Smith et al., “Similarities Among a Group of Elite Maize Inbreds as Measured by Pedigree, F₁ Grain Yield, Heterosis, and RFLPs,” Theor. Appl. Genet. 80:833-840 (1990), which is hereby incorporated by reference in its entirety) that each of the lines are different and can be uniquely identified on the basis of genetically-controlled molecular markers.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Elite inbred lines, that is, pure breeding, homozygous inbred lines, can be used as starting materials for breeding or as source populations from which to develop other inbred lines. These inbred lines derived from elite inbred lines can be developed using the pedigree breeding and recurrent selection breeding methods described earlier. As an example, when backcross breeding is used to create these derived lines in a melon plant breeding program, elite inbreds can be used as a parental line or starting material or source population and can serve as either the donor or recurrent parent.

Descriptions of other well known breeding methods that are commonly used for different traits and crops, and which can be used in combination with GSB resistant melons, including without limitation the GSB Resistant Melons of the present invention, can be found in one of several reference books (e.g., Allard, R. W., Principles of Plant Breeding, New York, N.Y.: John Wiley & Sons, Inc. (1960); Simmonds, N. W., Principles of Crop Improvement, New York, N.Y.: Longman (1979); Sneep and Hendriksen, eds., Plant Breeding Perspectives, Wageningen, The Netherlands: PUDOC (1979); Fehr, W. R., ed., Principles of Cultivar Development, Vol. 1: Theory and Technique, New York, N.Y.: Macmillan Publishing Company (1987); and Fehr, W. R., ed., Principles of Cultivar Development, Vol. 2: Crop Species, New York, N.Y.: Macmillan Publishing Company (1987), which are hereby incorporated by reference in their entirety).

In addition to traditional plant breeding techniques, melon breeding lines and cultivars may be developed using plant tissue culture techniques well known in the art. The term “tissue culture,” as used in the present application, is meant to be broad in scope and related to the culture of plant seeds, organs, explants, tissues, cells, or protoplasts in vitro or under sterile conditions. Tissue culture techniques include, but are not limited to, the following: micropropagation; meristem culture; somatic embryogenesis; somaclonal variation; in vitro selection; protoplast culture; somatic hybridization; and double-haploid breeding systems.

Various tissue culture techniques have been shown to produce plant germplasm with enhanced disease resistance (Daub, “Tissue Culture and the Selection of Resistance to Pathogens,” Ann. Rev. Phytopatholgy 24:159-186 (1986) and Hammerschlag, “In Vitro Approaches to Disease Resistance,” in Collins et al., eds., Applications of Genetic Engineering to Crop Improvement, Dordrecht: Matinus Nijhoff/Dr. W. Junk Publishers, pp. 453-490 (1984), which are hereby incorporated by reference in their entirety). Further, successful tissue cultures of melon using a variety of techniques have been reported in the art (Mackay et al., “Cucumis melo L. Callus Response to Toxins Produced by Myrothecium roridum Tode ex. Fries,” J. Amer. Soc. Hort. Sci. 119(2):356-360 (1994); Debeaujon et al., “Somatic Embryogenesis and Organogenesis from Protoplast-Derived Cultures of Muskmelon (Cucumis melo L.),” Acta Hort. (ISHS) 289:225-226 (1991); and Garcia-Sogo et al., “Enhancement of Morphogenetic Response in Cotyledon-Derived Explants of Cucumis melo Induced by Copper Ion,” Acta Hort. (ISHS) 289:229-230 (1991), which are hereby incorporated by reference in their entirety).

The use of double-haploid breeding systems has also been used in tissue cultures of melon. One method includes the use of parthenogenetic embryos of melon. In this procedure, two-week-old seedlings from melon plants are transplanted to containers (e.g., pots) and grown under favorable growth conditions in a greenhouse. The male flowers are collected on at least three different days, one of which is the day before anthesis, around 12 noon. The term “anthesis” refers to the period during which a flower opens or during the act of a flower opening, i.e., coming to full bloom. The male flowers are then irradiated with 250 Gray (25,000 Rad) in a gamma cell with Cesium source, and stored at room temperature. On the following day, female flowers are pollinated with the irradiated pollen or with pollen from control (non-irradiated) flowers. If the plant is monoecious, then it is not necessary to emasculate the maternal parent; however, their stigmas are covered with a capsule after pollination. Each female flower is then pollinated with at least three males.

Fruits that set are then harvested approximately 21-23 days after pollination and stored at room temperature before use (i.e., up to 5 days). Some fruits are sterilized with 20% Clorox and opened aseptically in a laminar flow hood. The seeds are excised, wiped on sterile paper towels, and opened individually to remove any embryos that are visible. These embryos are then cultured in E20A medium (Sauton et al., “Obtention De Plantes Haploïds Chez Melon (Cucumis melo L.) Par Gynogenèse Induite Par Du Pollen Irradiè,” Agronomie 7:141-148 (1987), which is hereby incorporated by reference in its entirety). Seeds from other sterilized fruits are wiped on sterile towels, washed in sterile water, and placed unopened in a 100×20 petri dish containing 20 ml of E20A liquid medium (about 80 seeds per dish). Dishes are sealed with Parafilm® (American National Can Co., Greenwich, Conn.) and incubated on a gyratory shaker (100 rpm) in light conditions (16 h day length). After about 10 days, the seeds that appear to contain embryos are opened aseptically. Embryos are excised and transferred to solid E20 for approximately 3 weeks to ensure that all embryos are rescued. In addition, seeds removed from some unsterilized fruits are rinsed in running water, sterilized for 10 minutes with 20% Clorox, and put into plates of E20A liquid medium. Cultured embryos that grow well are transferred to E20A solid medium. This can be done, for example, in baby food type jars with transparent caps. Subsequently, each plantlet is propagated to produce 3-6 new plants by putting the shoot tip and single nodes from the stem into fresh medium. Rooted plants are then transplanted to soil (e.g., mixture of peat moss and vermiculite) pots. After acclimatization, the plants are transferred to a greenhouse.

For comparison of gynogenesis with the use of irradiated pollen in producing haploid melon plants, female flowers of the maternal plants are collected at anthesis. Whole ovaries are dipped in Tween 20, left under running water for several minutes, sterilized (e.g., in Clorox for 15 minutes), and then rinsed three times in sterile distilled water. Each ovary (ca. 8-15 mm long) is cut into 6-10 cross-sections and cultured in 100×15 petri dishes on medium containing MS salts, vitamins, and 4% sucrose, solidified with 0.8% phytagar. The ovary slices are initially cultured for four or five days on the same medium containing 0.09 μM TDZ and then transferred to medium supplemented with 0.27 μM NAA and 0.88 μM BA in place of TDZ. The culture conditions used are the same as those used for the embryo cultures.

A deposit of a representative sample of seeds of a GSB resistant breeding line produced according to the methods of the present invention, and designated as NY 01-190-3R, -7L, -9L (composite), were deposited with the American Type Culture Collection (ATCC), 10801 University Blvd., Manassas, Va. 20110-2209. The date of submission of the deposit to ATCC was Nov. 13, 2001. The accession number for those deposited seeds of NY 01-190-3R, -7L, 9L (composite) is ______. The deposit will be maintained in the depository for a period of 30 years, or 5 years after the last request, or for the effective life of the patent, whichever is longer, and will be replaced if necessary during that period.

### EXAMPLES

### Example 1

### Identification of Gummy Stem Blight Resistant Cucumis melo Accessions

A rapid and reliable large-scale greenhouse screening procedure was used to evaluate gummy stem blight (“GSB”) resistance in approximately 798 U.S.D.A. Plant Introduction (“PI”) accessions. The materials and methods for the screening procedure were reported in Zhang et al., “Screening Melon (Cucumis melo) for Resistance to Gummy Stem Blight in the Greenhouse and Field,” HortScience 32:117-121 (1997), which is hereby incorporated by reference in its entirety. Of the PI accessions that were determined to exhibit GSB resistance, five PIs exhibiting high GSB resistance were selected for further evaluation and/or confirmation with regard to their mode of GSB resistance inheritance. These highly resistant PIs included: PI 157082; PI 511890; PI 482399; PI 482398; and PI 140471 (collectively referred to herein as the “GSB Resistant Melons”). To determine mode of GSB resistance inheritance, the GSB Resistant Melons were crossed with a susceptible melon parent. Analysis of GSB resistance in the F₁, F₂, and backcross populations of the crosses were used to determine whether resistance was due to recessive or dominant genes and whether the genes were monogenic or otherwise. Results from these analyses are reported in the examples below, portions of which were reported in Zuniga et al., “Monogenic Dominant Resistance to Gummy Stem Blight in Two Melon (Cucumis melo) Accessions,” Plant Dis. 83:1105-07 (1999), which is hereby incorporated by reference in its entirety.

### Example 2

### Analysis of Gummy Stem Blight Resistance in PI 157082, PI 511890, and PI 140471

The first objective was to determine the mode of inheritance of GSB resistance found in two highly resistant C. melo accessions, namely, PI 157082 and PI 511890. The second objective was to determine the genetic relationship between these sources of resistance and PI 140471, a wild melon previously described as having GSB resistance (Sowell et al., “Resistance of Cucumis melo Introductions to Mycosphaerella citrullina,” Plant Dis. Rep. 50:661-663 (1996), which is hereby incorporated by reference in its entirety) controlled by a single dominant gene (Prasad et al., “Inheritance of Resistance to Mycospaerella citrullina in Muskmelon,” J. Amer. Soc. Hortic. Sci. 91:396-400 (1967), which is hereby incorporated by reference in its entirety).

### Example 3

### Germplasm and Population Development of PI 157082, PI 511890, and PI 140471

C. melo PI 157082 and PI 511890 obtained from the U.S.D.A. National Plant Germplasm System at Ames, Iowa, were crossed with a susceptible genotype, ‘Cornell ZPPM 339’, with each other, and with PI 140471, to determine the mode of inheritance of GSB resistance. Cv. Cornell ZPPM 339 is a cantaloupe breeding line, well-adapted to greenhouse and field conditions, and monoecious, which eliminates the need for emasculation when used as a female parent. Controlled pollinations were carried out in the field and greenhouse to generate reciprocal F₁, F₂, and backcross (“BC”) progenies. Due to poor adaptation of PI 511890 to conditions in Ithaca, N.Y., and extreme susceptibility to powdery mildew, adequate seed were not able to be produced for testing from PI 157082×PI 511890 and reciprocal crosses, and from PI 511890×PI 140471 and reciprocal crosses, in either the greenhouse or the field. To confirm previously published results regarding genetics of resistance for PI 140471, PI 140471 was crossed with the cv. Mainstream, released by the United States Vegetable Laboratory in Charleston, S.C. (Nugent et al., “‘Mainstream’ Melon,” HortScience 14:192 (1979), which is hereby incorporated by reference in its entirety) and developed reciprocal F₁, F₂, and BC progenies. Cv. Mainstream was bred under unprotected conditions in South Carolina, and some tolerance to the disease was observed under some field conditions, but it was rated susceptible in inoculated tests (Zhang et al., “Screening Melon (Cucumis melo) for Resistance to Gummy Stem Blight in the Greenhouse and Field,” HortScience 32:117-121 (1997), which is hereby incorporated by reference in its entirety).

For field plantings in Ithaca, N.Y., 4-week-old seedlings from the greenhouse were acclimated for 3 days in cold frames outside the greenhouse before transplanting to beds covered with black plastic mulch. Plots consisted of 12 plants, with 2 plants per hill. Rows were spaced 2.1 m apart and hills were 0.6 m apart within the rows. Prior to transplanting, field plots were plowed and disked once, then broadcast fertilized with 225 Kg of 10-20-20 fertilizer. At transplant, approximately 500 ml of a starter fertilizer (9-45-15) was applied at a rate of 7 g/liter for each hill.

### Example 4

### Resistance Evaluations of PI 157082, PI 511890, and PI 140471

To assure uniform stands for resistance evaluations, seeds were germinated on paper towels held at 28° C. for 48 h, and seedlings were transplanted to Todd planter flats (4 by 8 cells; Hummert International, Earth City, Mo.) filled with Peatlite mix (1:1 peat moss:vermiculite). Two susceptible control plants each of ‘Top Mark’ and ‘Honeydew Greenflesh’ were planted randomly in each flat to monitor inoculation efficacy and test severity. Seedlings were held in a greenhouse at 24° C. All evaluations for genetic studies were carried out in the greenhouse essentially as described in Zhang et al., “Screening Melon (Cucumis melo) for Resistance to Gummy Stem Blight in the Greenhouse and Field,” HortScience 32:117-121 (1997), which is hereby incorporated by reference in its entirety, using NY1, a highly virulent D. bryoniae isolate collected in the field in Onondaga, N.Y. (Keinath et al., “Morphological, Pathological, and Genetic Differentiation of Didymella bryoniae and Phoma spp. Isolated from Cucurbits,” Phytopathology 85:364-369 (1995), which is hereby incorporated by reference in its entirety).

### Example 5

### Data Collection and Analyses of PI 157082, PI 511890, and PI 140471

Disease severity on stems of individual plants was recorded using a modified version of the procedure described in Zhang et al., “Screening Melon (Cucumis melo) for Resistance to Gummy Stem Blight in the Greenhouse and Field,” HortScience 32:117-121 (1997), which is hereby incorporated by reference in its entirety. Instead of a single reading at 7 days after inoculation, a second reading was also made at 21 days after inoculation. A 1 to 5 scale was used where 1=no damage; 2=a single lesion 1 to 10 mm long or coalesced lesions 1 to 20 mm, no girdling; 3=lesions 21 to 80 mm, girdling of the stem, or both; 4=withered stem; and 5=dead seedling (St. Amand et al., “Eight Isolates of Didymella bryoniae from Geographically Diverse Areas Exhibit Variation in Virulence But No Isolate by Cultivar Interaction on Cucumis sativus,” Plant Dis. 79:1136-1139 (1995), which is hereby incorporated by reference in its entirety). The numbers of individuals falling into resistant and susceptible classes were tallied, and observed segregation ratios in F₂ and BC populations were tested for goodness-of-fit using the chi-square statistic.

### Example 6

### Parental Reactions Regarding Analyses of PI 157082, PI 511890, and PI 140471

Disease scores for parental lines inoculated with the GSB pathogen are summarized in Table 1.

TABLE 1
Stem disease severity scores for melon genotypes used as parent in
this study 21 days after inoculation with Didymella bryoniae.

| Parents | Disease index^{a} | Rating |
|---|---|---|
| ZPPM 339 | 4.3 (±0.51) | Susceptible |
| PI 140471 | 1.1 (±0.20) | Resistant |
| PI 157082 | 1.3 (±0.57) | Resistant |
| PI 511890 | 1.3 (±0.47) | Resistant |

^{a}Mean ± standard deviation of severity scores based on a 1 to 5 scale (1 = no damage; 2 = a single lesion 1 to 10 mm long or coalesced lesions 1 to 20 mm, no girdling; 3 = lesion 21 to 80 mm, girdling of the stem, or both; 4 = withered stem; and 5 = dead seedling).

There was a clear and consistent difference in stem disease ratings at 21 days after inoculation between the resistant and susceptible parents in all tests. This defined the categories which were then used to group individuals from segregating populations. All plants of the resistant parents showed disease scores of 1 or 2. Almost all (96%) plants of PI 140471, 75% of PI 157082, and 70% of PI 511890 had scores of 1. The susceptible parent ZPPM 339 showed only disease scores between 3 and 5 (Table 1); 97% of these seedlings had either withered stems or were dead (scores of 4 and 5).

### Example 7

### Inheritance of Resistance in PI 157082

The reciprocal F₁ progenies between PI 157082 and ZPPM 339 showed uniformly high resistance, indistinguishable from the resistant parent, and consistent with dominant inheritance with no cytoplasmic (maternal) factors involved. The F₂ population segregated consistent with a 3:1 resistant:susceptible ratio (Table 2), supporting the hypothesis that resistance in this PI is controlled by a single dominant gene. Plants from the BC population derived from the resistant parent were uniformly resistant, and the BC derived from the susceptible parent segregated consistent with a 1:1 resistant:susceptible ratio (Table 2), which further supports monogenic dominant inheritance of gummy stem blight resistance from PI 157082.

TABLE 2
Response of Cucumis melo parental genotypes and intercrossed
populations to inoculation with Didymella bryoniae.

| | | Number of plants^{a} | | Expected | |
|---|---|---|---|---|---|
| Pedigree^{b} | Generation^{c} | R | S | ratio (R:S) | P_{a=0.05} |
| ZM | P | 1 | 30 | 0:1 | — |
| 471 | P | 24 | 0 | 1:0 | — |
| 082 | P | 20 | 0 | 1:0 | — |
| 890 | P | 26 | 0 | 1:0 | — |
| 082 × ZM | F₁ | 31 | 4 | 1:0 | — |
| ZM × 082 | F₁ | 14 | 2 | 1:0 | — |
| 082 × ZM | F₂ | 84 | 21 | 3:1 | 0.31 |
| (082 × ZM) × | BCₛ | 36 | 35 | 1:1 | 0.92 |
| ZM | | | | | |
| (082 × ZM) × | BCᵣ | 65 | 0 | 1:0 | — |
| 082 | | | | | |
| 890 × ZM | F₁ | 17 | 1 | 1:0 | — |
| ZM × 890 | F₁ | 46 | 1 | 1:0 | — |
| 890 × ZM | F₂ | 63 | 27 | 3:1 | 0.14 |
| (890 × ZM) × | BCₛ | 40 | 27 | 1:1 | 0.15 |
| ZM | | | | | |
| (890 × ZM) × | BCᵣ | 54 | 0 | 1:0 | — |
| 890 | | | | | |
| 471 × 082 | F₁ | 8 | 0 | 1:0 | — |
| 471 × 082 | F₂ | 234 | 23 | 15:1 | 0.13 |

^{a}R = resistant, S = susceptible.
^{b}ZM = ZPPM 339; 471 = PI 140471; 082 = PI 157082; and 890 = PI 511890.
^{c}P = parent; BCᵣ and BCₛ = backcross to resistant and susceptible parents, respectively.

### Example 8

### Relationship of Dominant Resistance from PI 157082 to the Mc (=Gsb1) Gene from PI 140471

To determine whether the dominant resistance allele in PI 157082 is related to the Mc locus previously identified by Prasad and Norton (Prasad et al., “Inheritance of Resistance to Mycospaerella citrullina in Muskmelon,” J. Amer. Soc. Hortic. Sci. 91:396-400 (1967), which is hereby incorporated by reference in its entirety), PI 157082 was crossed with PI 140471. The Mc locus is renamed herein as the Gsb1 locus. By producing and screening F₁, F₂, and resistant and susceptible BC progenies with C. melo cv. Mainstream, PI 140471 was confirmed to carry a single dominant gene. All plants from the F₁ population obtained by crossing PI 140471 and PI 157082 were uniformly highly resistant, with disease scores of 1 (Table 2). The F₂ plants segregated consistent with a 15:1 resistant:susceptible ratio (Table 2), supporting the hypothesis that GSB resistance in PI 157082 is controlled by a resistance allele at a locus that is unlinked to the Mc (=Gsb1) locus. Moreover, most of the plants in the susceptible category were dead (60%) or had severe stem infections (26%), while the majority (67%) of the resistant F₂ plants had scores of 1.

### Example 9

### Inheritance of Resistance in PI 511890

Reciprocal F₁ populations of PI 511890 and ZPPM 339 were highly resistant and their reactions did not differ, suggesting that GSB resistance from this source is also dominant, and that no cytoplasmic (maternal) factors are involved in its expression. F₂ plants segregated consistent with a 3:1 resistant:susceptible ratio (Table 2), indicating this source also possesses monogenic dominant resistance to GSB. As expected under this hypothesis, plants from the resistant BC were uniformly resistant, while those from the susceptible BC segregated consistent with a 1:1 resistant:susceptible ratio (Table 2).

Previously, a single dominant gene (Mc, also referred to herein as Gsb1) in PI 140471 was reported (Prasad et al., “Inheritance of Resistance to Mycospaerella citrullina in Muskmelon,” J. Amer. Soc. Hortic. Sci. 91:396-400 (1967), which is hereby incorporated by reference in its entirety) and has been used in the development of several varieties and breeding lines (Norton, J. D., “Gulfcoast—A Sweet Cantaloupe for the Produce Chain Store Market,” Ala. Agric. Exp. Stn. Leafl. 82 (1971); Norton, J. D., “Chilton, a High Quality Fruit for the Commercial Market,” Ala. Agric. Exp. Stn. Leafl. 84 (1972); Norton et al., “Ac-70- 154 a Gummy Stem Blight-Resistant Muskmelon Breeding Line,” HortScience 24:709-711 (1989); Norton et al., “Aurora—A High Quality Disease Resistant Cantaloupe,” Ala. Agric. Exp. Stn. Circ. 278 (1985), which are hereby incorporated by reference in their entirety). In a later study, however, releases originating from PI 140471 showed intermediate resistance or were rated as susceptible in greenhouse and field screens (Zhang et al., “Screening Melon (Cucumis melo) for Resistance to Gummy Stem Blight in the Greenhouse and Field,” HortScience 32:117-121 (1997), which is hereby incorporated by reference in its entirety). Zhang et al. suggested that this failure to recover parental levels of resistance in cultivated breeding types might be the result of interactions of the Mc (=Gsb1) gene with different genetic backgrounds. Alternatively, the resistance conferred by this gene may not be sufficiently high to protect vines that are under extreme physiological stress due to the concentrated set of large fruit (Zhang et al., “Screening Melon (Cucumis melo) for Resistance to Gummy Stem Blight in the Greenhouse and Field,” HortScience 32:117-121 (1997), which is hereby incorporated by reference in its entirety).

Results from genetic studies, using a different susceptible parent and a very reliable, severe screening method, confirmed the previous report of a single gene conferring resistance to GSB in PI 140471, namely, the Gsb1 gene. Similarly, using the same procedures, GSB resistance in PIs 157082 and 511890 were confirmed to be controlled by separate single dominant genes, Gsb2 and Gsb4, respectively, which genes were also distinct from the Gsb1. Preliminary results from the breeding program indicate that the combination of at least two different resistance genes may yield higher levels of resistance than either of the genes alone.

A survey has not been conducted of D. bryoniae isolates on these two new sources of resistance, so it is unknown if these genetic resources differ from PI 140471 with regard to fungal isolate specificity. It is known from previous research (Keinath et al., “Morphological, Pathological, and Genetic Differentiation of Didymella bryoniae and Phoma spp. Isolated from Cucurbits,” Phytopathology 85:364-369 (1995), which is hereby incorporated by reference in its entirety) and from additional studies that race-specificity does not exist in cucurbit species, including cucumber (St. Amand et al., “Eight Isolates of Didymella bryoniae from Geographically Diverse Areas Exhibit Variation in Virulence But No Isolate by Cultivar Interaction on Cucumis sativus,” Plant Dis. 79:1136-1139 (1995), which is hereby incorporated by reference in its entirety), although one previous report indicated that in melon, resistance from PI 140471 may not be consistently expressed in the field (Sowell, G., Jr., “Additional Sources of Resistance to Gummy Stem Blight of Muskmelon,” Plant Dis. 65:253-254 (1981), which is hereby incorporated by reference in its entirety). When combined in a single genotype, diverse sources of resistance may increase both the level of resistance and the breadth of protection, augmenting chemical disease control approaches which are currently judged inadequate (Keinath, A. P., “Fungicide Timing for Optimum Management of Gummy Stem Blight Epidemics on Watermelon,” Plant Dis. 79:354-358 (1995); and Keinath et al., “First Report on Benomyl-Insensitive Didymella bryoniae in the United States.,” (Abstr.) Phytopathology 85:1126 (1995), which are hereby incorporated by reference in their entirety), especially when environmental conditions are conducive to disease development (Keinath, A. P., “Fungicide Timing for Optimum Management of Gummy Stem Blight Epidemics on Watermelon,” Plant Dis. 79:354-358 (1995), which is hereby incorporated by reference in its entirety).

### Example 11

### Further Analyses of Gummy Stem Blight Resistance in PI 157082, PI 511890, PI 482399, PI 482398, and PI 140471, and Their Use in Breeding Programs

The GSB Resistant Melons—i.e., PI 157082, PI 511890, PI 482399, PI 482398, and PI 140471—were used as GSB resistance source plants in a breeding program in order to develop new GSB resistant melon hybrids. The non-resistant recurrent parents included, without limitation, Cornell ZPPM 339, TAM Uvalde, UC Topmark, Galia type, Ananas type, and Oro Rico. The breeding results indicate that the highest levels of resistance are achieved when the distinct resistance genes from the GSB Resistant Melons are combined.

To evaluate GSB resistance, all subject seedlings were inoculated in the greenhouse according to the standard protocol described in Zhang et al., “Screening Melon (Cucumis melo) for Resistance to Gummy Stem Blight in the Greenhouse and Field,” HortScience 32:117-121 (1997) and in Zuniga et al., “Monogenic Dominant Resistance to Gummy Stem Blight in Two Melon (Cucumis melo) Accessions,” Plant Dis. 83:1105-07 (1999), which are hereby incorporated by reference in their entirety. The seedlings were then transplanted to the field. In order to increase screening capacity, a mist table was used instead of a mist chamber. The mist table was used in accordance with well known methods in the art. Only resistant individuals of the more inbred advanced lines were taken to the field.

Genetic studies have defined several clear-cut sources of monogenic resistance to GSB. The original GSB source locus from PI 140471 was originally named Mc after a now-defunct name of the pathogen (Norton, J. D., “Gulfcoast—A Sweet Cantaloupe for the Produce Chain Store Market,” Ala. Agric. Exp. Stn. Leafl. 82 (1971); Norton, J. D., “Chilton, a High Quality Fruit for the Commercial Market,” Ala. Agric. Exp. Stn. Leafl. 84 (1972); Norton et al., “Ac-70- 154 a Gummy Stem Blight-Resistant Muskmelon Breeding Line,” HortScience 24:709-711 (1989); Norton et al., “Aurora—A High Quality Disease Resistant Cantaloupe,” Ala. Agric. Exp. Stn. Circ. 278 (1985), which are hereby incorporated by reference in their entirety). As previously described herein, the Mc gene locus has been renamed Gsb1.

In addition to the Gsb1 gene locus, at least three other independent dominant, monogenic resistance genes have been identified using the inheritance study techniques describe herein. The three additional dominant, monogeneic resistance genes are as follows: Gsb2, from PI 157082; Gsb4, from PI 511890; and Gsb5, from PI 482398. Inheritance studies also confirmed that resistance from PI 482399 is monogenic recessive, and this locus has been designated gsb3. All five resistance genes—namely, Gsb1, Gsb2, gsb3, Gsb4, and Gsb5—have been shown to be separate and distinct from one another.

In addition to the genetic studies described above, molecular markers (RAPDS, initially) linked to these loci are being investigated. Tissue has been harvested from the appropriate segregating F2 populations used in the allelism studies for each of the GSB Resistant Melon sources of resistance in order to use bulk segregant analysis to identify tightly linked molecular markers for the purpose of pyramiding these genes.

For breeding, at least 2,100 plants were screened for GSB resistance in inoculated greenhouse tests as previously described herein. The tests consisted of 4 TAM Uvalde BCF2 populations, 11 Cornell ZPPM 339 BCF2 populations, and 50 selected F1 progenies from both backgrounds. Resistant selections were selfed, backcrossed, and intercrossed.

Field tests were conducted for 36 F3 individuals that were derived from resistant selected F2 plants, 4 TAM Uvalde backcross progenies, 5 Cornell ZPPM 339 BC progenies, and 16 intercross progenies. The F3 individuals, GSB Resistant Melon PIs, and Norton's varieties (i.e., Gulfcoast PVP 616B Aurora” and/or “Chilton PVP 616D 89A-25 Norton”) were inoculated in the greenhouse and in the field. Commercial checks were only inoculated in the field. The field inoculation was applied with a backpack sprayer (spore concentration 100-200,000 spores/ml). Ideally, field inoculation was applied on an evening that proceeds a day that is cloudy or damp. The plants were only inoculated once because of the severity of the initial greenhouse screen and the fact that well-established symptoms are visible in the field. Susceptible check rows were inoculated with the inbred lines from the greenhouse inoculation. The check plants that survived the greenhouse screen were all diseased and unproductive.

Populations inbred to the F5 and F6 generations from the original crosses have been produced. Emphasis has been on intercrossing populations that derive their resistance from different PIs. The recurrent parents used were Cornell ZPPM 339, TAM Uvalde, and UC Topmark. Many of the best plants combine resistance from PI 140471 and PI 511890. Progenies derived from the African PIs (i.e., PI 482399 and PI 482398) have a tendency towards yellow crowns. The amount fruit set is not indicative of overall yield due to stripping open-pollinated fruit from plants to promote fruit.

The GSB Resistant Melon PIs were acquired directly from the USDA-ARS Plant Introduction Station, I.S.U., Ames, Iowa 50011, Attn. Linda Minor.

The Recurrent parents, commercial varieties, used for the field inoculations included the following:

(1) 00-601 Mainstream 602 Perlita;

(2) 603 Athena 604 Durango;

(3) 605 Cordelle 606 Quasar;

(4) 607 Vienna 608 Hales Best Jumbo;

(5) 609 Honeyrock 610 Earlyqueen;

(6) 611 Alienor 612 TAM Uvalde;

(7) 613 Topmark 614 ZPPM339; and

(8) 615 Honeydew Green flesh.

The resistant varieties/accessions used in the breeding program included:

(1) 00-616A Gulfcoast PVP 616B Aurora; and

(2) 616C Chilton PVP 616D 89A-25 Norton.

The resistant parents used in the plant breeding program included:

(1) 00-617A PI 157082 (China);

(2) 617B PI 482399 (Zimbabwe);

(3) 617C PI 140471 (Texas);

(4) 617D PI 482398 (Zimbabwe); and

(5) 617E PI 511890 (Mexico).

TAM Uvalde has been emphasized as a recurrent parent because of its combining ability for use in Eastern and Western hybrids and some indication that it already has some field tolerance to GSB. Although its fruits are small in size, TAM Uvalde has commercially appealing attributes and has a very attractive interior. Mainstream was used in early crosses for the same reason, but has not combined well for quality and so emphasis has shifted to TAM Uvalde, Cornell ZPPM339, and UC Topmark as recurrent parents. This material has also been crossed to some cucumber mosaic virus (“CMV”) resistant material from Europe. The BC progenies in rows 680-685 have this and/or Cornell CMV tolerant breeding lines in their pedigrees. A Galia type adapted to Egypt has been used as another recurrent parent for backcrossing.

The rows included in the above-referenced experiment were as follows:

Rows 00-618-626: Advanced TAMUvalde lines that performed well in the greenhouse screen;

Rows 00-627-653: Advanced ZPPM339 lines that performed well in greenhouse screen;

Rows 00-654-669: R×R F1s in the ZPPM339 background;

Rows 00-670-679: BCF1s in both TAM Uvalde and ZPPM339 backgrounds; and

Rows 00-680-685: Crosses to CMV resistant material.

The following non-resistant, commercially available melon varieties may also be used as non-recurrent parents in the methods of the present invention: Galia type (Hollar Seed Co., Rocky Ford, Colo.); Ananas type (Hollar Seed Co., Rocky Ford, Colo.); and Oro Rico (Harris Moran Seed Company, Modesto, Calif.).

A summary of the breeding program performed using the resistant and non-resistant melons described herein is as follows: In field tests, emphasis was placed on self-pollination on resistant cross-resistant F₁s and BC F₁s. From these selfed progenies, 27 F₂ lines were chosen to screen in greenhouse tests. These 27 families were selected primarily on the basis of their levels of GSB resistance. However, an additional selection criterion was large fruit size. Sixty plants per family were planted for a total of 1,620 F₂ plants. A total of 117 plants were saved, about 7 percent of the starting population. This percentage was consistent with predicted amounts expected to be saved based on information gathered from the inheritance of resistance studies. Self-pollinations were successfully obtained in the greenhouse on most of these plants and F₃ seed are being harvested for field planting. Based on the results of the F₂ screen, 10 of the most resistant F₂ lines were selected for rescreening and for inclusion in another field planting. Approximately 60 plants per F₂ line, for a total of 600 plants, were planted. Also, 53 F₄ lines, 2 F₅ lines, and 2 F₆ lines were planted in the field. The selection of these particular advanced lines was based primarily on GSB resistance. About 30 plants per line, for a total of 1,710 plants, were planted. The F₂ and advanced lines have been inoculated in the greenhouse and have been selected for resistance. Selections are to be planted in the field, where they will be inoculated again to ensure adequate disease pressure. Emphasis will be placed on self-pollinations and crosses between resistant plants with complementary types, especially those that bring together different resistance genes. Additional non-resistant, recurrent parents, including, without limitation, Galia type, Ananas type, and Oro Rico, will be used with the GSB Resistant Melons to backcross resistance into the non-resistant types.

As is readily apparent to one skilled in the art, the foregoing are only some of the various ways by which the inbred can be obtained by those looking to use the germplasm. Other means are available, and the above examples are illustrative only.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for producing a gummy stem blight resistant Cucumis melo hybrid seed comprising:
crossing a first Cucumis melo plant with a second Cucumis melo plant to yield a first generation hybrid seed, wherein said first plant is either resistant to gummy stem blight or not resistant to gummy stem blight and the second plant is resistant to gummy stem blight.

2. The method according to claim 1, wherein the first Cucumis melo plant is not resistant to gummy stem blight and is selected from the group consisting of C. melo cantalupensis, C. melo indorus, and C. meloflexuousus.

3. The method according to claim 1, wherein the first Cucumis melo plant is not resistant to gummy stem blight and is selected from the group consisting of Cornell ZPPM 339, TAM Uvalde, UC Topmark, Galia type, Ananas type, and Oro Rico.

4. The method according to claim 1, wherein the first Cucumis melo plant is resistant to gummy stem blight and is selected from the group consisting of U.S.D.A. Plant Introduction (“PI”) accession PI 157082, PI 511890, PI 482399, PI 482398, and PI 140471.

5. The method according to claim 1, wherein the second Cucumis melo plant is selected from the group consisting of U.S.D.A. Plant Introduction (“PI”) accession PI 157082, PI 511890, PI 482399, PI 482398, and PI 140471.

6. The method according to claim 1, wherein the first Cucumis melo plant is resistant to gummy stem blight and contains at least one resistance gene for gummy stem blight.

7. The method according to claim 6, wherein said resistance gene is selected from the group consisting of a dominant resistance gene and a recessive resistance gene.

8. The method according to claim 7, wherein said resistance gene is a dominant resistance gene selected from the group consisting of Gsb1, Gsb2, Gsb4, and Gsb5.

9. The method according to claim 7, wherein said resistance gene is a gsb3 recessive resistance gene.

10. The method according to claim 1, wherein the second Cucumis melo plant contains at least one resistance gene for gummy stem blight.

11. The method according to claim 10, wherein said resistance gene is selected from the group consisting of a dominant resistance gene and a recessive resistance gene.

12. The method according to claim 11, wherein said dominant resistance gene is selected from the group consisting of Gsb1, Gsb2, Gsb4, and Gsb5.

13. The method according to claim 11, wherein said resistance gene is a gsb3 recessive resistance gene.

14. The method according to claim 1, wherein both said first Cucumis melo plant and said second Cucumis melo plant contain at least one resistance gene for gummy stem blight.

15. The method according to claim 14, wherein said resistance gene is selected from the group consisting of a dominant resistance gene and a recessive resistance gene.

16. The method according to claim 14, wherein said dominant resistance gene is selected from the group consisting of Gsb1, Gsb2, Gsb4, and Gsb5.

17. The method according to claim 14, wherein said resistance gene is a gsb3 recessive resistance gene.

18. The method according to claim 1 further comprising:
growing the first generation hybrid seed to yield a first generation resistant Cucumis melo hybrid plant.

19. The method according to claim 18 further comprising:
using germplasm derived from the hybrid plant in a plant breeding program to yield gummy stem blight resistant Cucumis melo hybrid seeds.

20. The method according to claim 19, wherein said breeding program comprises traditional plant breeding techniques selected from the group consisting of pedigree breeding, selling, intercrossing, and backcrossing.

21. The method according to claim 19, wherein said breeding program comprises tissue culture techniques.

22. A gummy stem blight resistant Cucumis melo hybrid seed, prepared by a method comprising:
crossing a first Cucumis melo plant with a second Cucumis melo plant to yield a first generation hybrid seed, wherein said first plant is either resistant to gummy stem blight or not resistant to gummy stem blight and the second plant is resistant to gummy stem blight.

23. The gummy stem blight resistant Cucumis melo hybrid seed according to claim 22, wherein the first Cucumis melo plant is not resistant to gummy stem blight and is selected from the group consisting of C. melo cantalupensis, C. melo indorus, and C. meloflexuousus.

24. The gummy stem blight resistant Cucumis melo hybrid seed according to claim 22, wherein the first Cucumis melo plant is not resistant to gummy stem blight and is selected from the group consisting of Cornell ZPPM 339, TAM Uvalde, UC Topmark, Galia type, Ananas type, and Oro Rico.

25. The gummy stem blight resistant Cucumis melo hybrid seed according to claim 22, wherein the first Cucumis melo plant is resistant to gummy stem blight and is selected from the group consisting of U.S.D.A. Plant Introduction (“PI”) accession PI 157082, PI 511890, PI 482399, PI 482398, and PI 140471.

26. The gummy stem blight resistant Cucumis melo hybrid seed according to claim 22, wherein the second plant is a gummy stem blight resistant Cucumis melo plant selected from the group consisting of U.S.D.A. Plant Introduction (“PI”) accession PI 157082, PI 511890, PI 482399, PI 482398, and PI 140471.

27. The gummy stem blight resistant Cucumis melo hybrid seed according to claim 22, wherein the first Cucumis melo plant contains a resistance gene for gummy stem blight.

28. The gummy stem blight resistant Cucumis melo hybrid seed according to claim 27, wherein said resistance gene is selected from the group consisting of a dominant resistance gene and a recessive resistance gene.

29. The gummy stem blight resistant Cucumis melo hybrid seed according to claim 28, wherein said dominant resistance gene is selected from the group consisting of Gsb1, Gsb2, Gsb4, and Gsb5.

30. The gummy stem blight resistant Cucumis melo hybrid seed according to claim 28, wherein said resistance gene is a gsb3 recessive resistance gene.

31. The gummy stem blight resistant Cucumis melo hybrid seed according to claim 22, wherein the second Cucumis melo plant contains a resistance gene for gummy stem blight.

32. The gummy stem blight resistant Cucumis melo hybrid seed according to claim 31, wherein said resistance gene is selected from the group consisting of a dominant resistance gene and a recessive resistance gene.

33. The gummy stem blight resistant Cucumis melo hybrid seed according to claim 32, wherein said dominant resistance gene is selected from the group consisting of Gsb1, Gsb2, Gsb4, and Gsb5.

34. The gummy stem blight resistant Cucumis melo hybrid seed according to claim 32, wherein said resistance gene is a gsb3 recessive resistance gene.

35. The gummy stem blight resistant Cucumis melo hybrid seed according to claim 22, wherein both said first Cucumis melo plant and said second Cucumis melo plant contain at least one resistance gene for gummy stem blight.

36. The gummy stem blight resistant Cucumis melo hybrid seed according to claim 35, wherein said resistance gene is selected from the group consisting of a dominant resistance gene and a recessive resistance gene.

37. The gummy stem blight resistant Cucumis melo hybrid seed according to claim 36, wherein said dominant resistance gene is selected from the group consisting of Gsb1, Gsb2, Gsb4, and Gsb5.

38. The gummy stem blight resistant Cucumis melo hybrid seed according to claim 36, wherein said resistance gene is a gsb3 recessive resistance gene.

39. A gummy stem blight resistant Cucumis melo hybrid plant, or its parts, produced by the seed of claim 22.

40. Pollen of the hybrid plant according to claim 39.

41. An ovule of the hybrid plant according to claim 39.

42. A tissue culture of regenerable cells of the hybrid plant according to claim 39, wherein the tissue culture regenerates multiple progeny plants capable of expressing all the morphological and physiological characteristics of said hybrid plant.

43. A gummy stem blight resistant Cucumis melo progeny plant, or its parts, regenerated from the tissue culture of claim 42.

44. A gummy stem blight resistant Cucumis melo offspring plant, or its parts, wherein at least one ancestor of said offspring plant is the hybrid plant, or its parts, of claim 39.

45. A gummy stem blight resistant Cucumis melo hybrid plant, prepared by a method comprising:
crossing a first Cucumis melo plant with a second Cucumis melo plant to yield a first generation hybrid seed, wherein said first plant is either resistant to gummy stem blight or not resistant to gummy stem blight and said second plant is resistant to gummy stem blight; and
growing the first generation hybrid seed to yield a first generation resistant Cucumis melo hybrid plant.

46. The gummy stem blight resistant Cucumis melo hybrid plant according to claim 45, wherein the first Cucumis melo plant is not resistant to gummy stem blight and is selected from the group consisting of C. melo cantalupensis, C. melo indorus, and C. meloflexuousus.

47. The gummy stem blight resistant Cucumis melo hybrid plant according to claim 45, wherein the first Cucumis melo plant is not resistant to gummy stem blight and is selected from the group consisting of Cornell ZPPM 339, TAM Uvalde, UC Topmark, Galia type, Ananas type, and Oro Rico.

48. The gummy stem blight resistant Cucumis melo hybrid plant according to claim 45, wherein the first Cucumis melo plant is resistant to gummy stem blight and is selected from the group consisting of U.S.D.A. Plant Introduction (“PI”) accession PI 157082, PI 511890, PI 482399, PI 482398, and PI 140471.

49. The gummy stem blight resistant Cucumis melo hybrid plant according to claim 45, wherein the second plant is a gummy stem blight resistant Cucumis melo plant selected from the group consisting of U.S.D.A. Plant Introduction (“PI”) accession PI 157082, PI 511890, PI 482399, PI 482398, and PI 140471.

50. The gummy stem blight resistant Cucumis melo hybrid plant according to claim 45, wherein the first Cucumis melo plant is resistant to gummy stem blight and contains at least one resistance gene for gummy stem blight.

51. The gummy stem blight resistant Cucumis melo hybrid plant according to claim 50, wherein said resistance gene is selected from the group consisting of a dominant resistance gene and a recessive resistance gene.

52. The gummy stem blight resistant Cucumis melo hybrid plant according to claim 51, wherein said dominant resistance gene is selected from the group consisting of Gsb1, Gsb2, Gsb4, and Gsb5.

53. The gummy stem blight resistant Cucumis melo hybrid plant according to claim 51, wherein said resistance gene is a gsb3 recessive resistance gene.

54. The gummy stem blight resistant Cucumis melo hybrid plant according to claim 45, wherein the second Cucumis melo plant is resistant to gummy stem blight and contains at least one resistance gene for gummy stem blight.

55. The gummy stem blight resistant Cucumis melo hybrid plant according to claim 54, wherein said resistance gene is selected from the group consisting of a dominant resistance gene and a recessive resistance gene.

56. The gummy stem blight resistant Cucumis melo hybrid plant according to claim 55, wherein said dominant resistance gene is selected from the group consisting of Gsb1, Gsb2, Gsb4, and Gsb5.

57. The gummy stem blight resistant Cucumis melo hybrid plant according to claim 55, wherein said resistance gene is a gsb3 recessive resistance gene.

58. The gummy stem blight resistant Cucumis melo hybrid plant according to claim 45, wherein both said first Cucumis melo plant and said second Cucumis melo plant contain at least one resistance gene for gummy stem blight.

59. The gummy stem blight resistant Cucumis melo hybrid plant according to claim 58, wherein said resistance gene is selected from the group consisting of a dominant resistance gene and a recessive resistance gene.

60. The gummy stem blight resistant Cucumis melo hybrid plant according to claim 59, wherein said dominant resistance gene is selected from the group consisting of Gsb1, Gsb2, Gsb4, and Gsb5.

61. The gummy stem blight resistant Cucumis melo hybrid plant according to claim 59, wherein said resistance gene is a gsb3 recessive resistance gene.

62. Pollen of the resistant hybrid plant according to claim 45.

63. An ovule of the resistant hybrid plant according to claim 45.

64. A tissue culture of regenerable cells of the hybrid plant according to claim 45, wherein the tissue culture regenerates multiple progeny plants capable of expressing all the morphological and physiological characteristics of said hybrid plant.

65. A gummy stem blight resistant Cucumis melo progeny plant, or its parts, regenerated from the tissue culture of claim 64.

66. A gummy stem blight resistant Cucumis melo offspring plant, or its parts, wherein at least one ancestor of said offspring plant is the hybrid plant, or its parts, of claim 45.

67. Seed of a gummy stem blight resistant Cucumis melo breeding line designated NY 01-190-3R, -7L, -9L (composite), a sample of said seed having been deposited under ATCC accession number ______.

68. A Cucumis melo plant, or its parts, produced by the seed of claim 67.

69. Pollen of the plant of claim 68.

70. An ovule of the plant of claim 68.

71. A tissue culture of regenerable cells of the Cucumis melo breeding line according to claim 67, wherein the tissue culture regenerates multiple progeny plants capable of expressing all the morphological and physiological characteristics of said hybrid Cucumis melo breeding line.

72. A tissue culture according to claim 71, wherein the tissue is selected from the group consisting of leaves, pollen, embryos, roots, stems, root tips, anthers, flowers, seeds, and fruit.

73. A Cucumis melo plant, or its parts, regenerated from the tissue culture of claim 71 and capable of expressing all the morphological and physiological characteristics of said Cucumis melo breeding line.
